(19) United States
(12) Patent Application Publication
(10) Pub. No.: US 2024/0342016 A1
(43) Pub. Date: Oct. 17, 2024

(54) POSTPARTUM UNDERGARMENT APPARATUS

(71) Applicants: William Weise, Mc Kinney, TX (US); Rhonda Weise, Mc Kinney, TX (US)

(72) Inventors: William Weise, Mc Kinney, TX (US); Rhonda Weise, Mc Kinney, TX (US)

(21) Appl. No.: 18/133,587

(22) Filed: Apr. 12, 2023

(51) Int. Cl. A61F 13/496; A61F 13/49; A61F 13/494

(52) U.S. Cl. CPC A61F 13/496; A61F 13/49011; A61F 13/49473; A61F 2013/1517; A61F 2013/49088

(57) ABSTRACT

A postpartum undergarment apparatus for absorbing bodily fluids and facilitating healing after childbirth includes a garment which is worn around a hip of a user. The garment includes an absorbent member which is positioned to absorb vaginal blood that is released from wounds sustained during childbirth. The garment also includes a waistband which is stretchable so that it applies a compression force on a torso of the user near a fundus of a uterus of the user when worn. The compression force aids the uterus in contracting after childbirth and may accelerate a recovery process of the user.

### (1) FIELD OF THE INVENTION

The disclosure relates to postpartum garments and more particularly pertains to a new postpartum garment for absorbing bodily fluids and facilitating healing after childbirth.

### (2) DESCRIPTION OF RELATED ART INCLUDING INFORMATION DISCLOSED UNDER 37 CFR 1.97 AND 1.98

The prior art discloses several postpartum garments for absorbing vaginal blood and other bodily fluids which discharge after childbirth. The prior art also includes postpartum garments which apply a compressive force near a caesarean section incision to reduce inflammation and promote healing at the site of the incision. However, the prior art fails to disclose a postpartum garment which absorbs vaginal blood released by a user's body after childbirth while providing an elastic compress which facilitates reduction of the size of a uterus after childbirth. Such a garment is desirable, because it provides sufficient leak protection to the user while also aiding the user's body in reducing the size of the uterus. Without compression, uteri typically contract naturally, though the experience can be more uncomfortable for the user.

### BRIEF SUMMARY OF THE INVENTION

An embodiment of the disclosure meets the needs presented above by generally comprising a hip portion has a tubular shape and is configured for is worn around a hip of a user. An absorbent member is coupled to and positioned at a bottom side of the hip portion and is configured for absorbing vaginal blood. A waistband is coupled to a top end of the hip portion and is resiliently stretchable. When the apparatus is worn, the waistband is stretched around a torso of the user such that a compression force is exerted by the waistband on the torso. The waistband is configured for being positioned over a fundus of a uterus of the user such that the compression force is exerted onto the fundus.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a postpartum undergarment apparatus according to an embodiment of the disclosure.

FIG. 2 is a front view of an embodiment of the disclosure.

FIG. 3 is a side view of an embodiment of the disclosure.

FIG. 4 is a top view of an embodiment of the disclosure.

FIG. 5 is a cross-section view of an embodiment of the disclosure taken from Arrows 5-5 in FIG. 2.

FIG. 6 is an in-use view of an embodiment of the disclosure.

### DETAILED DESCRIPTION OF THE INVENTION

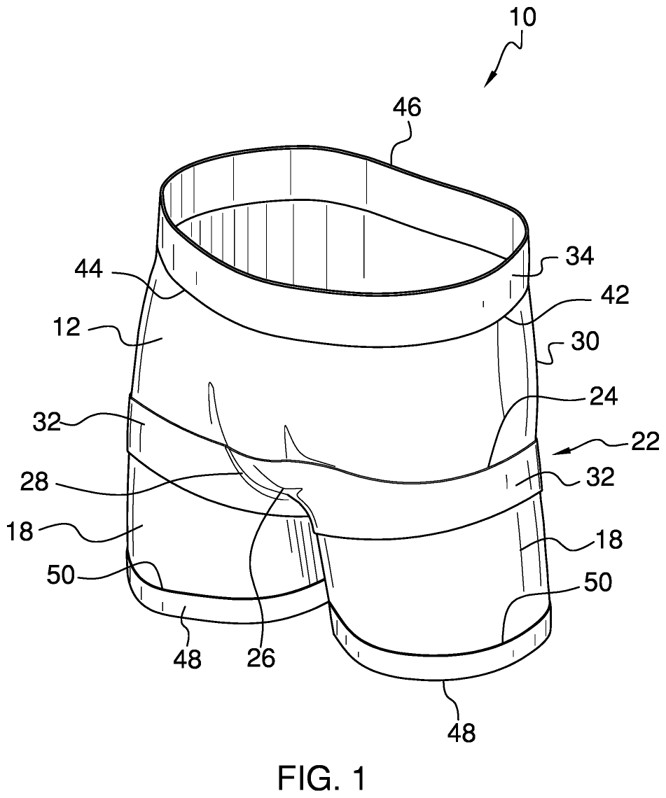

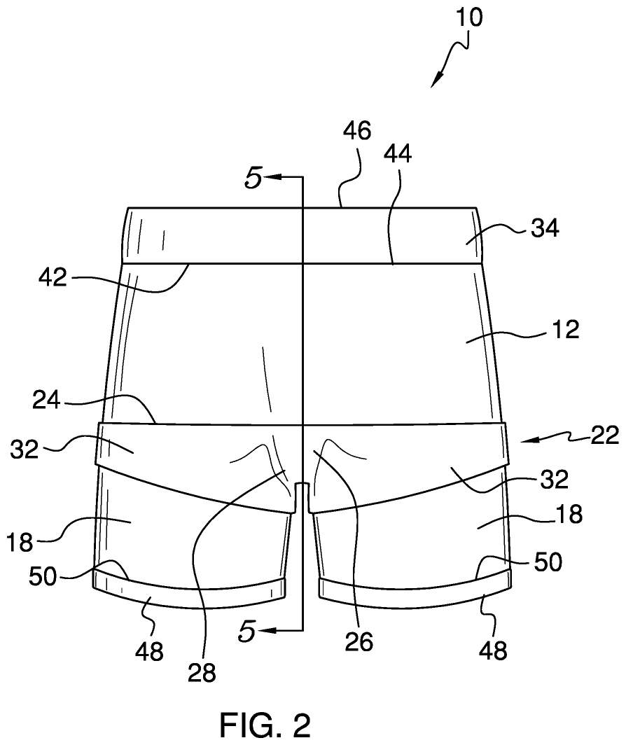

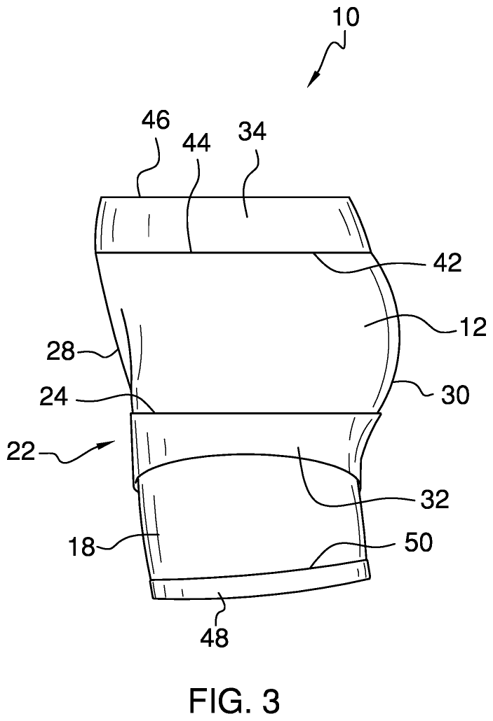

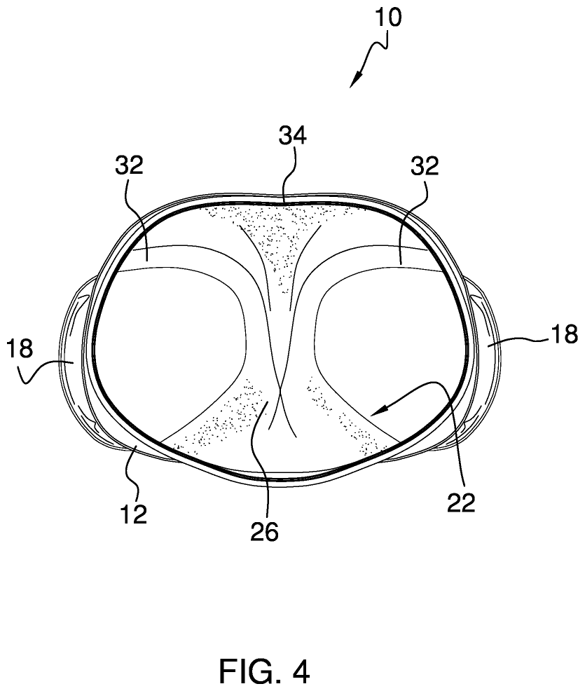

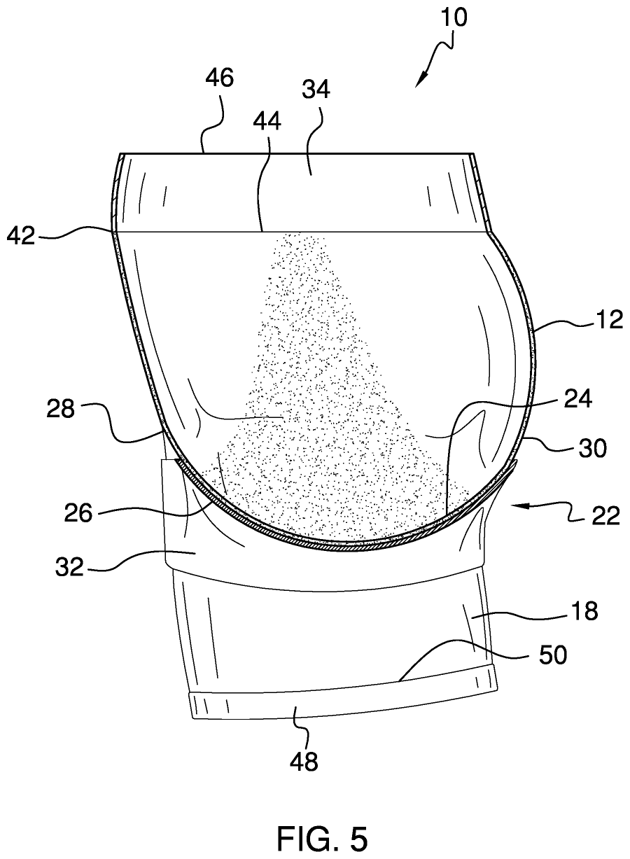

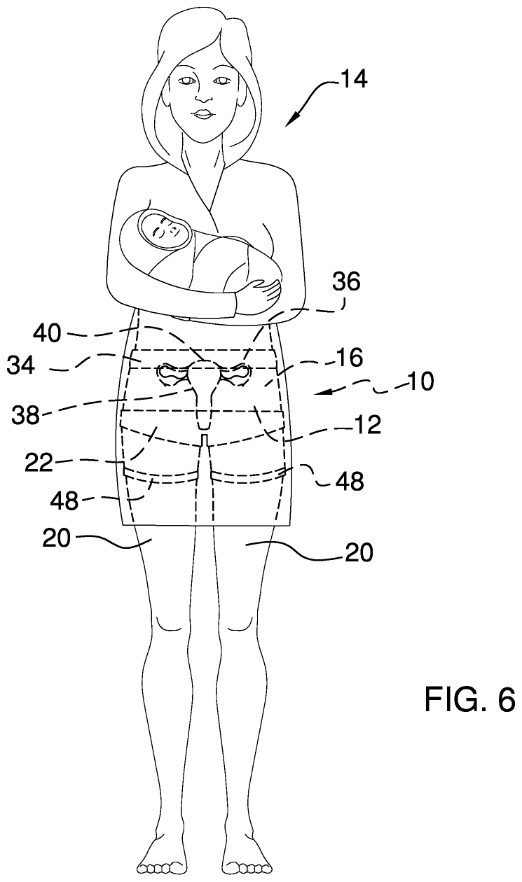

With reference now to the drawings, and in particular to FIGS. 1 through 6 thereof, a new postpartum garment embodying the principles and concepts of an embodiment of the disclosure and generally designated by the reference numeral 10 will be described.

As best illustrated in FIGS. 1 through 6, the postpartum undergarment apparatus generally comprises a hip portion 12 with a tubular shape and which is configured for being worn around a hip 16 of a user 14. A pair of leg portions 18 is coupled to and extends downwardly from the hip portion 12. Each leg portion 18 of the pair of leg portions 18 has a tubular shape and is configured for being worn around a respective one of a pair of legs 20 of the user 14.

An absorbent member 22 is coupled to and positioned at a bottom side 24 of the hip portion 12. The absorbent member 22 comprises an absorbent material which is configured for absorbing vaginal blood, which is often released from wounds sustained by the user 14 after the user 14 has given vaginal birth. The absorbent member 22 has a crotch portion 26 extending from a front side 28 of the hip portion 12 to a rear side 30 of the hip portion 12 along the bottom side 24 and between the pair of leg portions 18. The absorbent member 22 also includes a pair of lateral portions 32, each of which extends around a respective leg portion 18 of the pair of leg portions 18. The absorbent member 22 may comprise cotton, wool, bamboo, sodium polyacrylate, potassium polyacrylate, or the like.

A waistband 34 is coupled to a top end 42 of the hip portion 12 and is resiliently stretchable. The waistband 34 is configured for being stretched around a torso 36 of the user 14 such that a compression force is exerted by the waistband 34 on the torso 36. The waistband 34 is configured for being positioned over a fundus 40 of a uterus 38 of the user 14 such that the compression force is exerted onto the fundus 40. The compression force assists the uterus 38 to contract after childbirth. The waistband 34 has a width of at least 2.75 inches between a proximal edge 44 of the waistband 34 and a distal edge 46 of the waistband 34 with respect to the hip portion 12. Each leg band 48 of a pair of leg bands 48 is coupled to a lower end 50 of an associated leg portion 18 of the pair of leg portions 18. Each leg band 48 is resiliently stretchable such that each leg band 48 forms a seal with an associated leg 20 of the pair of legs 20 to prevent leakage of the vaginal blood out of the postpartum garment apparatus.

In use, the postpartum garment apparatus is worn by the user 14 after the user 14 gives birth. The waistband 34 is stretched around the torso 36 of the user 14 and positioned over the fundus 40 of the uterus 38 of the user 14 so that the waistband 34 exerts the compression force onto the fundus 40. The absorbent member 22 is positioned below the vagina of the user 14 so that the vaginal blood may be absorbed. Other bodily fluids may also be absorbed by the absorbent member 22, and blood resulting from menstruation and medical procedures such as caesarean sections may also be absorbed by the absorbent member 22.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A postpartum undergarment apparatus comprising:
a hip portion having a tubular shape and being configured for being worn around a hip of a user;
an absorbent member being coupled to and positioned at a bottom side of the hip portion, the absorbent member comprising an absorbent material configured for absorbing vaginal blood; and
a waistband being coupled to a top end of the hip portion, the waistband being resiliently stretchable, the waistband being configured for being stretched around a torso of the user such that a compression force is exerted by the waistband on the torso, the waistband being configured for being positioned over a fundus of a uterus of the user such that the compression force is exerted onto the fundus.

2. The apparatus of claim 1, wherein the waistband has a width of at least 2.75 inches between a proximal edge of the waistband and a distal edge of the waistband with respect to the hip portion.

3. The apparatus of claim 1, further comprising a pair of leg portions being coupled to and extending downwardly from the hip portion, each leg portion of the pair of leg portions having a tubular shape and being configured for being worn around a respective one of a pair of legs of the user, the absorbent member having a crotch portion extending from a front side of the hip portion to a rear side of the hip portion along the bottom side and between the pair of leg portions.

4. The apparatus of claim 3, wherein the absorbent member further comprises a pair of lateral portions, each lateral portion of the pair of lateral portions extending around a respective leg portion of the pair of leg portions.

5. The apparatus of claim 1, further comprising a pair of leg bands, each leg band of the pair of leg bands being coupled to a lower end of an associated leg portion of the pair of leg portions, each leg band being resiliently stretchable such that each leg band forms a seal with an associated leg of the pair of legs to prevent leakage of the vaginal blood out of the postpartum garment apparatus.

6. A postpartum undergarment apparatus comprising:
a hip portion having a tubular shape and being configured for being worn around a hip of a user;
a pair of leg portions being coupled to and extending downwardly from the hip portion, each leg portion of the pair of leg portions having a tubular shape and being configured for being worn around a respective one of a pair of legs of the user;
an absorbent member being coupled to and positioned at a bottom side of the hip portion, the absorbent member comprising an absorbent material configured for absorbing vaginal blood, the absorbent member having a crotch portion extending from a front side of the hip portion to a rear side of the hip portion along the bottom side and between the pair of leg portions, the absorbent member further comprising a pair of lateral portions, each lateral portion of the pair of lateral portions extending around a respective leg portion of the pair of leg portions;
a waistband being coupled to a top end of the hip portion, the waistband being resiliently stretchable, the waistband being configured for being stretched around a torso of the user such that a compression force is exerted by the waistband on the torso, the waistband being configured for being positioned over a fundus of a uterus of the user such that the compression force is exerted onto the fundus, the waistband having a width of at least 2.75 inches between a proximal edge of the waistband and a distal edge of the waistband with respect to the hip portion; and
a pair of leg bands, each leg band of the pair of leg bands being coupled to a lower end of an associated leg portion of the pair of leg portions, each leg band being resiliently stretchable such that each leg band forms a seal with an associated leg of the pair of legs to prevent leakage of the vaginal blood out of the postpartum garment apparatus.
